(19) United States
(12) Patent Application Publication
(10) Pub. No.: US 2007/0224616 A1
(43) Pub. Date: Sep. 27, 2007

(54) METHOD FOR FORMING MOLECULAR SEQUENCES ON SURFACES

(76) Inventors: Erdogan Gulari, Ann Arbor, MI (US); Jean-Marie Rouillard, Ann Arbor, MI (US); Xiaolian Gao, Houston, TX (US); Xiaochuan Zhou, Houston, TX (US)

Correspondence Address: JULIA CHURCH DIERKER;DIERKER & ASSOCIATES, P.C., 3331 W. BIG BEAVER RD. SUITE 109, TROY, MI, 48084-2813, US

(21) Appl. No.: 11/690,368

(22) Filed: Mar. 23, 2007

Related U.S. Application Data
(60) Provisional application No. 60/785,938, filed on Mar. 24, 2006.

(51) Int. Cl. C12Q 1/68; B05D 3/00

(52) U.S. Cl. 435/6; 427/2.11

(57) ABSTRACT

A method for forming molecular sequences includes derivatizing an unconfined substrate surface with at least one linker containing a protected reactive group. The substrate is contacted with a solution containing a photogenerated reagent precursor and a buffer and/or a neutralizer. A photogenerated reagent is generated in at least a portion of the solution. The photogenerated reagent is configured to initiate the formation of at least one active region on the substrate surface. A monomer is coupled to the active region.

### CROSS-REFERENCE TO RELATED APPLICATIONS

This application claims the benefit of U.S. Provisional Patent Application Ser. No. 60/785,938 filed Mar. 24, 2006, which is incorporated herein by reference in its entirety.

### STATEMENT REGARDING FEDERALLY SPONSORED RESEARCH OR DEVELOPMENT

This invention was made in the course of research partially supported by grants from the National Institutes of Health (NIH), Grant Nos. 1R01RR018625-01 and 1R21HG003725-01. The U.S. government has certain rights in the invention.

### BACKGROUND

The present disclosure relates generally to methods for forming molecular sequences on surfaces.

High density microarrays of biopolymers on solid surfaces, or biochips for diagnostic and research purposes have been shown to have great potential. Biochips (including DNA biochips, protein biochips, peptide biochips, and the like) containing in situ synthesized microarrays have been used in a variety of applications, including guiding patient care, monitoring progression of diseases through gene expression changes, identifying single nucleotide polymorphisms (SNPs), identifying the genetic reasons for many cancers, detecting viruses that infect the central nervous system, detecting and identifying pathogens, understanding the relationship between the songbird genomics and the learning patterns, developing drugs, and changing plant genetics in response to the environment.

Biochip fabrication includes direct on-chip synthesis (making several sequences at a time) involving inkjets; direct on-chip parallel synthesis (making the whole array of sequences simultaneously) involving photolithography and specially made molecules containing UV sensitive protection groups; direct on-chip parallel synthesis involving photogenerated acids and bases and arrays of pre-fabricated reaction wells in the substrate; and direct on-chip synthesis using electrochemically generated acids and immobilization of a library of pre-synthesized molecules involving robotic spotting.

Spotting and inkjet technologies can include additional steps that may, in some instances, be somewhat inefficient, complex, and relatively labor intensive. For example, spotting and inkjet techniques may include pre-synthesizing each molecular sequence separately before putting them on a substrate, repetitive micropipetting of the samples, and substrates that need micromachined chambers or special hydrophobic surface treatment for physical confinement of reactions.

Light directed on-chip parallel synthesis may include the following limitations: the chemistries often require specialized, costly, and difficult to synthesize, light cleavable protection groups on linkers and monomers used; and the synthesis may suffer from low sequence fidelity.

Many of the techniques for forming biochips include confining the synthesis areas by physical barriers, polymer matrices, or surface tension barriers. The addition of such barriers may require fabrication of three-dimensional synthesis chambers between two substrates using semiconductor manufacturing techniques, or hydrophobic surface patterning.

As such, it would be desirable to provide a synthesis method that is relatively simple, versatile, cost effective, and capable of producing high density molecular arrays of improved purity.

### SUMMARY

A method for forming molecular sequences is disclosed. The method includes derivatizing an unconfined substrate surface with at least one linker containing a protected reactive group. The substrate is contacted with a solution containing a photogenerated reagent precursor and a buffer and/or a neutralizer. A photogenerated reagent is generated in at least a portion of the solution. The photogenerated reagent is configured to initiate the formation of at least one active region on the substrate surface. A monomer is bound to the active region.

### BRIEF DESCRIPTION OF THE DRAWINGS

Features and advantages of embodiments of the present disclosure will become apparent by reference to the following detailed description and drawings, in which like reference numerals correspond to similar, though not necessarily identical components. For the sake of brevity, reference numerals or features having a previously described function may not necessarily be described in connection with other drawings in which they appear.

FIG. 1 is a schematic diagram of an embodiment of forming molecular sequences (SEQ ID NO: 1, SEQ ID NO: 2, SEQ ID NO: 3, SEQ ID NO: 4, and SEQ ID NO: 5 are shown as non-limiting example sequences);

FIG. 2 is a numerical simulation of the chemical confinement of photogenerated reagents;

FIG. 3 is a schematic diagram of an embodiment of forming a molecular sequence using a photogenerated acid precursor (SEQ ID NO: 6, SEQ ID NO: 7, and SEQ ID NO: 8 are shown as non-limiting example sequences);

FIG. 4 is a schematic diagram comparing a conventional solution-based acid deprotection reaction in an oligonucleotide synthesis with an embodiment of the photogenerated acid-based oligonucleotide synthesis;

FIG. 5 is a schematic diagram of an embodiment of forming molecular sequences using a photogenerated base precursor (SEQ ID NO: 9, SEQ ID NO: 10, and SEQ ID NO: 11 are shown as non-limiting example sequences);

FIG. 6 is a schematic diagram of an apparatus for synthesizing embodiments of molecular sequences;

FIG. 7A depicts oligonucleotide sequences formed on an unpatterned glass substrate;

FIG. 7B depicts oligonucleotide sequences formed on a glass microscope slide;

FIG. 7C depicts oligonucleotide sequences formed on a 200 micron silica sphere; and

FIG. 7D depicts oligonucleotide sequences formed on the inside walls of a capillary tube.

### DETAILED DESCRIPTION

Embodiments of the method disclosed herein advantageously allow the preparation of different chemical sequences at predetermined locations on a substrate surface without physical divisions, porous gel/polymer matrix patterning, or surface chemical treatments (e.g., hydrophobic or hydrophilic patterning). Furthermore the method(s) disclosed herein may be applied to prepare large scale arrays of DNA, RNA oligonucleotides, peptides, oligosaccharides, glycolipids, and other organic and biopolymers on a solid substrate. Embodiment(s) of the arrays formed herein may be used in a variety of chemical, biological, and/or medical applications. Examples of such applications include, but are not limited to screening for biological activities (e.g., drugs, antibodies), drug discovery, clinical diagnosis, gene expression analysis, genotyping, discovery of genetic mutations of living beings, subsequent sequencing, detection of single nucleotide polymorphisms, sequencing by hybridization, determination of promoter binding sites, polymerase chain reaction, epitope binding, ligand—peptide interaction, heavy metal detection, gene synthesis, protein DNA interaction, preparation of combinatorial libraries of polymeric molecules, and/or the like, and/or combinations thereof.

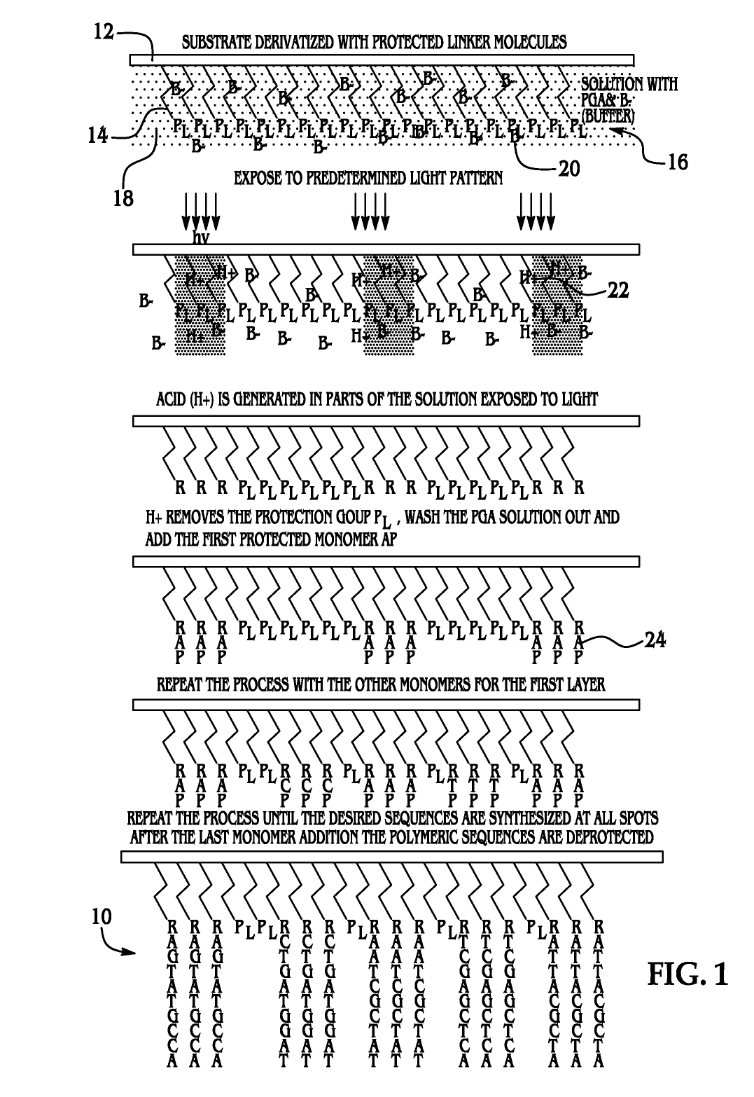

Referring now to FIG. 1, a schematic diagram of an embodiment of forming molecular sequences 10 is depicted. It is to be understood that the sequences 10 may be formed at predetermined regions of the substrate surface without using photolabile protecting groups, photomasks, or other means of physical confinement, such as surface tension, hydrophobic or hydrophilic barriers, microfabricated walls, etc. Sequences 10 that are formed via embodiments of the method may include, but are not limited to oligonucleotides, oligopeptides, polyesters, nylons, polyurethanes, polyamides, polycarbonates, oligosaccharides, and/or the like, and/or combinations thereof. In the embodiment shown in FIG. 1, oligonucletide sequences are formed.

In an embodiment, an unconfined substrate 12 surface is derivatized with at least one linker molecule 14. The substrate 12 is generally any solid or semisolid material, or a surface-coated solid material. In an embodiment, the surface of the substrate 12 is substantially flat, rounded (e.g., the inside of a capillary tube), composed of a layer of micro beads, the surface of microparticle(s) and/or nanoparticle(s) having an arbitrary shape, or combinations thereof. Non-limitative examples of suitable substrate materials include glass, quartz, silicon, silica spheres, porous glass, nylon sheets or membranes, TENTAGEL (TentaGel resins, commercially available from Rapp Polymere GmbH in Tübingen, Germany, are grafted copolymers including a low crosslinked polystyrene matrix on which polyethylene glycol (PEG or POE) is grafted), and/or the like, and/or combinations thereof.

It is to be understood that the linker molecule(s) 14 may be any molecule having an end capable of binding/bonding to the substrate 12 surface, and having another end that contains a protected reactive group. In an embodiment, the molecule(s) 14 bind/bond to the substrate 12 surface via a covalent bond, the multivalency effect, electrostatic attraction, complexation (e.g., thiol groups binding to gold surfaces), or the like, or combinations thereof. Non-limitative examples of the linker molecule(s) 14 include 3-aminopropyltrimethoxysilane, 3-aminopropyltriethoxy silane, 3-carboxypropyl silane, nucleophosphoramidites, nucleophosphonates (a non-limitative example of which includes 5′-Dimethoxytrityl-2′-deoxyThymidine,3′-[(2-cyanoethyl)-(N,N-diisopropyl)]-phosphoramidite), amino acids (a non-limitative example of which includes ter-butyloxycarbonyl (t-BOC) alanine), and/or the like, and/or combinations thereof.

In an embodiment, the reactive group of the linker molecule 14 is protected by an acid or base labile protection group P_{L}. Non-limitative examples of the labile protection group P_{L} include dimethoxytrityl (DMT), monomethoxytrityl (MMT), diesters, fluorenylmethyloxycarbonyl (Fmoc), t-BOC, benzyl-oxycarbonyl (CBZ), methoxyethylidene (MED), acetyl, trifluoro acetyl, esters and their derivatives, and/or the like, and/or combinations thereof.

The derivatized substrate 12 may be contacted with a solution 16 containing a photogenerated reagent precursor 18 and a buffer or a neutralizer 20. The solution 16 may also contain a sensitizer, a stabilizer, a viscosity additive, and/or combinations thereof.

It is believed that the sensitizer (e.g., photosensitizers) may increase the efficiency of the generation of the photogenerated reagent 22 (described further hereinbelow) and/or alter the wavelength at which the photogenerated reagent 22 is generated. Non-limitative examples of suitable photo sensitizers are anthracene, anthracene derivatives, dicyanoanthracene, thioxanthone, chlorothioxanthenes, pyrene, benzophenone, acetophenone, benzoinyl C1-C12 alkyl ethers, benzoyltriphenylphosphine oxide, Ru²⁺ complexes, Ru²⁺ complex derivatives, any chromophogenic compound, derivatives thereof, and/or the like, and/or combinations thereof Embodiments of the solution 16 including a sensitizer may also include an excited molecule trapper that substantially prevents diffusion of the sensitizer molecules away from illuminated sites (described further hereinbelow). Non-limiting examples of such molecules include molecular oxygen, mannitol, azide ion, GRP Carotenal (Girards reagent P derivative of beta-apo-8carotenal), carnosine (B-alanyl-L-histidine), cetylmethylviologen, triethanolamine, metallophorphyrins, A-tocopherol, B-carotene derivatives, and/or like, and/or combinations thereof.

Examples of stabilizers include, but are not limited to R—H stabilizers, non-limitative examples of which include propylene carbonate, propylene glycol ethers, t-butane, t-butanol, thiols, cyclohexane, substituted derivatives thereof, or combinations thereof. The substituted derivatives of these non-limitative examples include at least one of the following substituent groups: halogens, NO₂, CN, OH, SH, CF₃, C(O)H, C(O)CH₃, C₁-C₃-acyl, SO₂CH₃, C₁-C₃—SO₂R₂, OCH₃, SCH₃, C₁-C₃—OR₂, C₁-C₃—SR₂, NH₂, C₁-C₃—NHR₂, C₁-C₃—N(R₂)₂, (where R₂=alkyl group, which may be the same or a different group when present more than once in the compound), or the like.

Non-limitative examples of viscosity modifiers include glycerol, polyethylene glycol (PEG), polyvinyl pyrollidone (PVP), polyisobutane, polyacrylic acid, polymethylmethacrylate, derivatives thereof, or the like, or combinations thereof.

A photogenerated reagent precursor 18 is a precursor molecule that forms an acid or a base and a byproduct when exposed to electromagnetic radiation with sufficient energy to initiate the precursor's decomposition. The photogenerated reagent precursor 18 may be a photoacid generator (generates H⁺, in the form of an organic acid, a Lewis acid, or an inorganic acid) or a photobase generator (generates an organic base, a Lewis base, or an inorganic base).

Non-limitative examples of photoacid generator precursors include diazoketones, triarylsulfonium salts, iodinum salts, naphthalimide compounds, naphthalimide-oxy compounds, benzyloxycarbonyl compounds, phenylethoxycarbonyl compounds, phenylpropoxycarbonyl compounds, and/or the like, and/or combinations thereof. Specific examples of suitable photoacid generator precursors include, but are not limited to bis(4-tert-butylphenyl)iodonium perfluoro-1-butanesulfonate; bis(4-tert-butylphenyl)iodonium p-toluenesulfonate; bis(4-tert-butylphenyl)iodonium triflate; (4-Bromophenyl)diphenylsulfonium triflate; (tert-butoxycarbonylmethoxynaphthyl)-diphenylsulfonium triflate; (tert-butoxycarbonylmethoxyphenyl)diphenylsulfonium triflate; (4-tert-butylphenyl)diphenylsulfonium triflate; (4-chlorophenyl)diphenylsulfonium triflate; diphenyliodonium-9,10-dimethoxyanthracene-2-sulfonate; diphenyliodonium hexafluorophosphate; diphenyliodonium nitrate; diphenyliodonium perfluoro-1-butanesulfonate; diphenyliodonium p-toluenesulfonate; diphenyliodonium triflate; (4-fluorophenyl)diphenylsulfonium triflate; N-hydroxynaphthalimide triflate; N-hydroxy-5-norbornene-2,3-dicarboximide perfluoro-1-butanesulfonate; N-hydroxyphthalimide triflate; [4-[(2-hydroxytetradecyl)oxy]phenyl]phenyliodonium hexafluoroantimonate; (4-Iodophenyl)diphenylsulfonium triflate; (4-methoxyphenyl)diphenylsulfonium triflate; 2-(4-methoxystyryl)-4,6-bis(trichloromethyl)-1,3,5-triazine; (4-methylphenyl)diphenylsulfonium triflate; (4-methylthiophenyl)methyl phenyl sulfonium triflate; 2-naphthyl diphenylsulfonium triflate; (4-phenoxyphenyl)diphenylsulfonium triflate; (4-phenylthiophenyl)diphenylsulfonium triflate; thiobis(triphenyl sulfonium hexafluorophosphate)solution; triarylsulfonium hexafluoroantimonate salts; triarylsulfonium hexafluorophosphate salts; triphenylsulfonium perfluoro-1-butanesufonate; triphenylsulfonium triflate; tris(4-tert-butylphenyl)sulfonium perfluoro-1-butanesulfonate; tris(4-tert-butylphenyl)sulfonium triflate; and/or combinations thereof

Examples of photobase precursors include, but are not limited to o-benzocarbamates, benzoinlycarbamates, oxime urethanes, formanilides, dimethylbenzyl-oxycarbonylamines, benzyloxyamine derivatives, phenylethoxycarbonyl derivatives, any other molecule containing an amino or amine group protected by a photolabile group that is capable of releasing the amino or amine or a Lewis base upon exposure to light, and/or the like, and/or combinations thereof.

It is to be understood that the buffer or neutralizer 20 selected may be dependent upon, at least in part, the photogenerated reagent precursor 18 in the solution 16. Non-limitative examples of suitable buffers or neutralizers 20 for use with a photoacid precursor include pyridine, lutidine, piperidine, primary, secondary or tertiary amines or derivatives thereof, any organic base or Lewis base that is soluble in organic solvents (e.g., ammonia), and/or the like, and/or combinations thereof. In an embodiment in which a photobase precursor is used, the buffer or neutralizer 20 is selected from weak acids, Lewis acids soluble in organic solvents, and/or the like, and/or combinations thereof. Non limiting examples of such acids include benzillic acid, aluminum chloride, iron (III) chloride, boron trifluoride, ytterbium (III) triflate, butyric acid, propionic acid, phenol, and/or the like, and/or combinations thereof.

The substrate 12 and solution 16 are exposed to electromagnetic radiation (e.g., light) at predetermined area(s) such that a photogenerated reagent 22 is generated in the solution 16 at the predetermined area(s). It is to be understood that the predetermined area(s) may be any suitable size and/or shape that is determined, in part, by the optics used to expose the area to light.

The conditions at which the photogenerated reagent 22 is generated are generally mild (e.g., room temperature, neutral or mild solvents), and the reaction is relatively fast (e.g., seconds or fractions of a second).

It is believed that the buffer and/or neutralizer 20 present in the solution 16 react(s) with the photogenerated reagent 22, thereby forming a neutral species that is incapable of further reacting. The formation of the neutral species confines and restricts the action of the photogenerated reagent 22 to the substantially immediate neighborhood of the substrate 12 predetermined area(s). As such, the chemical activity of the photogenerated reagent 22 may be directed to predetermined locations on the substrate 12 surface, without the use of barriers, photomasks, hydrophobic patterning, or the like. It is believed that this buffer-reagent interaction increases the threshold of acid or base deprotection at areas where a fraction of the photogenerated reagent is activated. This results in improved contrast between the region receiving light irradiation, and the region receiving irradiation due to light dispersion.

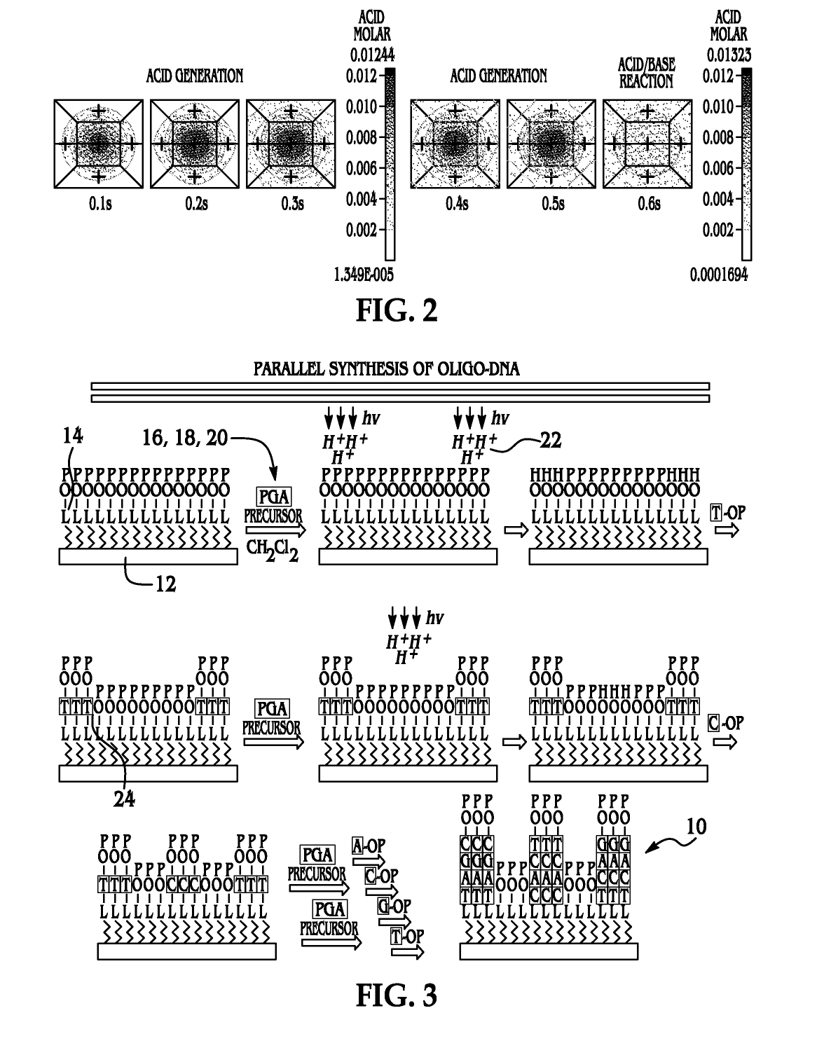

Numerical simulations of the buffer and/or neutralizer 20 and photogenerated reagent 22 reaction are shown in FIG. 2. The acid is substantially continuously generated from the precursor 18 for up to about 0.6 seconds, at which point the light exposure ceases. Generally, once light exposure ceases, the photogenerated reagent 22 concentration rapidly decreases and becomes essentially zero in a relatively short time period, for example, about two seconds. It is to be understood that the illustrated concentrations are at the surface of the substrate 12. As shown in each simulation, the area on the substrate 12 where acid generation occurs is circular, even though the light exposure is rectangular in shape. It is believed that this change occurs, at least in part, because of the higher availability of neutral species near the corners of the projected image.

The photogenerated reagent 22 may be an acid or a base depending, at least in part, on the photogenerated reagent precursor 18 selected. The photogenerated reagent 22 is also configured to initiate the formation of at least one active region on the substrate 12. After exposure to radiation, the photogenerated reagent 22 diffuses to the substrate 12 surface where it catalyzes the deprotection of the linker molecule(s) 14. The labile protection group P_{L} is removed to expose the reactive group(s) R within the predetermined areas and to form an active area.

The substrate 12 may be washed, and then contacted with a solution containing one or more monomers 24 and an activator. A monomer 24 is capable of coupling to each of the reactive group(s) R, and it is believed that the activator advantageously hastens this coupling reaction. Non-limitative examples of monomers 24 include nucleotides (DNA (e.g., C, T, A and G shown in the molecular sequences 10 of FIG. 1) or RNA (e.g., C, U, A and G)), locked nucleic acid (LNA) monomers, amino acids (peptides or proteins (e.g., Ala, Cys, Asp, etc.)), mono and disaccharides (such as, for example, glucose, sucrose, maltose and/or the like), and/or combinations thereof. It is to be understood that any of the monomers 24 may be natural, synthetic, or a combination of the two. Non-limitative examples of activators include tetrazole; 4,5, dicyanoimidazole (DCI); pyridiniumtrifluoroacetate; 5-ethlythiotetrazole; 5 (3,5-dintrophenyl)-1H-tetrazole; trimethylchlorosilane; activator 42 (5-(bis-3,5-trifluormethylphenyl)1-H-tetrazole; derivatives thereof, and/or the like; and/or combinations thereof.

It is to be understood that the monomer(s) 24 may have one end capable of coupling to the reactive group(s) R and another end that includes a reactive group protected by a protection group P (which may be the same as, or different from the labile protection group P_{L}). In one embodiment, the protected monomers 24 are used to synthesize known sequences. It is to be further understood, however, that the monomer(s) 24 may not include protection group P. In one embodiment, the unprotected monomers 24 are used to synthesize random sequences.

The process may be repeated as desired to de-protect linker(s) 14, monomer(s) 24, or combinations thereof, and to selectively couple additional monomer(s) 24 thereto to form desired sequences 10. In a non-limitative example, for an oligonucleotide biochip containing arrays of any designated sequence patterns, the maximum number of reaction steps is 4×1 in each cycle if natural nucleotides are used, and 12×1 if non-natural nucleobase-containing nucleotides are also used. Thus, the maximum number of cycles for synthesizing an oligonucleotide array of “n” nucleotides is 4×n if natural DNA monomers are used, and more if non-natural monomers are also used.

FIG. 3 depicts an embodiment of synthesizing an oligonucleotide sequence 10. Similar to FIG. 1, predetermined linker molecules 14 are deprotected after being contacted with a solution 16 and exposed to light. Various monomers 24 (e.g., nucleophosphoramidite monomers, T, A, C and G) are attached to active sites of the deprotected linker molecules 14.

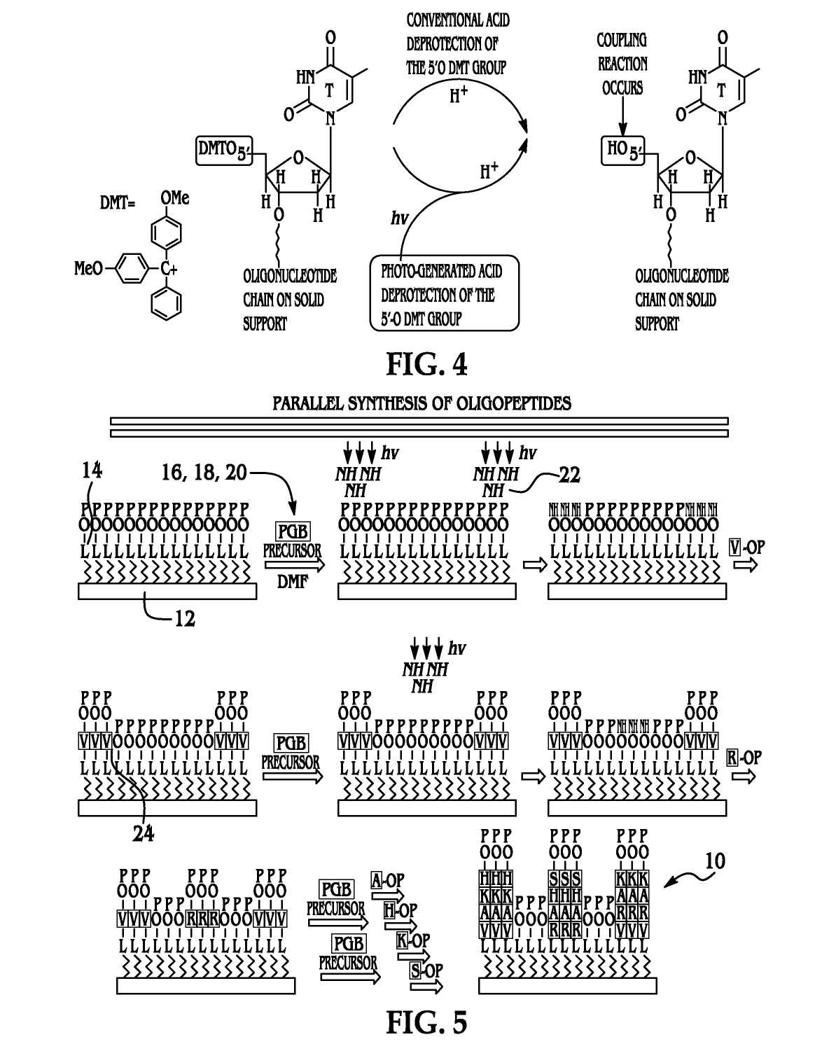

Referring now to FIG. 4, a solution-based acid deprotection reaction in an oligonucleotide synthesis is depicted. After acid is generated upon light exposure, the protecting groups (e.g., DMT) of the linkers are cleaved to expose reactive 5′-OH groups. A phosphite bond is capable of forming between the —OH groups of the linkers and reactive phosphorus atoms of monomers. Washing, oxidation, and capping steps of typical phosphoramidite or phosphonate synthesis processes may be performed, which would complete the addition of the first monomer.

In another embodiment, monomers containing protected 3′-OH groups may be used instead of the 5′-OH groups to carry out the synthesis of the oligonucleotides in the 3′ to 5′ direction. This type of synthesis may be used in synthesizing PCR primers.

Referring now to FIG. 5, a non-limitative example of synthesizing amino acid polymers (e.g., oligopeptides) in a parallel fashion on an open substrate 12 using the F-moc method is depicted. Protected linker molecules 14 are attached to the surface of the substrate 12. In this non-limiting example, each linker molecule 14 contains a reactive functional group (e.g., —NH₂) that is protected by a base labile protection group P, P_{L} (F-moc in this example).

It is to be understood that in the embodiments disclosed herein, the substrate 12 may be attached to a reactor cartridge, either at its bottom or top, such that the derivatized surface faces the inside of the cartridge.

The substrate 12 is then contacted by a solution 16 containing a photogenerated reagent precursor 18. In this example embodiment, the photogenerated reagent precursor 18 is a photobase generator selected from 2-nitrobenzyoxycarbonyl-piperidine, 2-nitrophenylpropoxycarbonyl, 5-benzyl-1,5-diazabicyclo-nonane, 5-benzyl-1,5-diazabicyclo-undecane, 5-benzyl-1,4-diazabicyclo-imidazole, and combinations thereof.

A predetermined light pattern is then projected onto the substrate 12 and the solution 16. The photogenerated reagent 22 is a base (such as, amines including, for example, piperidine, C₅H₁₁N (i.e., hexahydropyridine), pentamethyleneimine, azacyclohexane, 1,5-diazabicyclo-undecene (DBU), 5-benzyl-1,5-diazabicyclo-nonene (DBN), 1,4-diazabicyclo-imidazole, etc.) and is produced in the parts of the solution 16 exposed to light. The photogenerated reagent 22 removes the protection groups P, P_{L} from the linker molecules 14. In this non-limitative example, the removal of the protection groups P, P_{L} results in the exposure of reactive NH groups.

It is to be understood that the photogenerated reagent 22 is not generated in the solution 16 at area(s) that is/are not exposed to light, and diffusion of the photogenerated reagent 22 to the non-exposed sites is prevented by reaction with a neutralizing (in this example a weak acid) or buffering molecule present in the solution 16.

The substrate surface is washed and subsequently contacted with a solution of the first amino acid monomer 24 (a non-limitative example of which contains a reactive carboxylic acid group and a protected amine group), and a coupling agent/activator (e.g., carbodiimide-mediated coupling, benzotriazol-1-yloxy-tris(dimethylamino)phosphonium (BOP), O-benzotriazol-1-y1-N,N,N′,N′-tetramethyluronium hexafluorophosphate (HBTU), O-(7-azabenzotriazol-1-y1)-N,N,N′,N′-tetramethyluronium hexafluorophosphate (HATU), and/or the like). The amino acid monomer(s) 24 adds to the deprotected linker molecules 14 to produce an amide linkage.

In this example embodiment, the attached amino acid monomer(s) 24 contains a reactive functional terminal amine group protected by a base-labile group (e.g., F-moc). The substrate 12 surface may be contacted with a second batch of a solution 16 and exposed to a second predetermined light pattern. The monomer(s) 24 or linkers 14 in the exposed areas are deprotected, and the substrate 12 is washed and subsequently supplied with the second monomer (F-moc-R, where R may be any suitable amino acid). The second monomer attaches to the surface sites that have been deprotected by the light exposure.

As previously indicated, the synthesis may be repeated until polymers (e.g., a sequence of sequentially connected amino acids AHVSK (SEQ ID NO: 12)) of desired lengths and chemical sequences are formed at selected surface sites. It is to be understood that the number of cycles to add the desired amino acid monomers 24 to the predefined sites on the substrate 12 is generally less than or equal to 20×1 if naturally occurring amino acids and/or their derivatives are used, but may be significantly greater than 20×1 if non-natural amino acid analogues are also used.

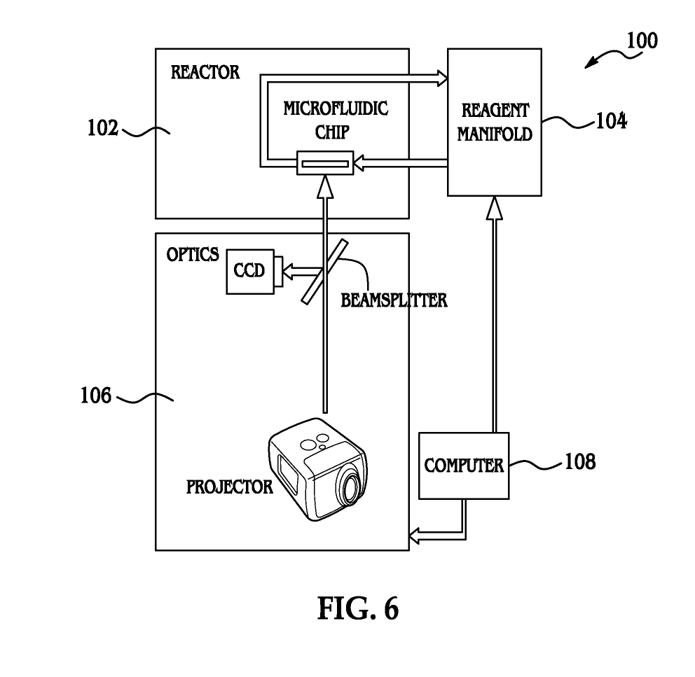

FIG. 6 illustrates an apparatus 100 for synthesizing large-scale sequences 10. Generally, the apparatus 100 includes a chemical reactor cartridge 102, a reagent manifold 104, an optical system 106, and a computer control system 108.

The chemical reactor 102 includes a housing with a manifold for bringing liquid reagents into contact with the substrate 12. In an embodiment, the chemical reactor 102 is machined or molded out of an inert material (non-limitative examples of which include fluorinated polymers, polyethylene, PEEK, stainless steel, and/or other suitable materials). The reactor 102 has an inlet and an outlet for feeding the reagents and washing solvents. In an embodiment, the reactor 102 may be heated and/or cooled by contacting with a heating and/or cooling source (e.g., IR, microwave, heating elements, cooling coils etc.).

A fractal manifold of two or more levels may be used, at least in part, to make the flow of the reagents over the substrate 12 uniform. The top and/or the bottom of the reactor is/are covered by the substrate(s) 12 on which the desired polymeric sequences will be synthesized in a predetermined pattern. Generally, the connection between the substrate 12 and the chemical reactor cartridge 102 is sealed with an o-ring or a gasket of appropriate material(s). In another embodiment, the flow guiding manifold is etched out of silicon, glass, or another inert ceramic material, and the substrate 12 is attached by either anodic bonding, diffusion bonding, or by the use of a gluing agent (e.g., an epoxy).

The reactor 102 is then connected to the reagent manifold by mounting the reactor 102 into a cartridge.

The reagent manifold 104 performs reagent metering, delivery, circulation, and disposal. Generally, the manifold 104 includes reagent bottles, solenoid or pressure actuated valves, metering pumps, inert gas handling system, tubing, and/or process controllers. It is to be understood that the reagent manifold 104 may be built separately, or a DNA/RNA, peptide, or other type of automated synthesizer may be used as reagent manifolds 104.

The optical system 106 generates predetermined patterns for light-directed synthesis. The optical system 106 includes a light source, a spatial light modulator, lenses, mirrors and/or filters. In an embodiment, the light source is a mercury UV lamp, a Xenon lamp, an incandescent lamp, a visible or UV laser, light emitting diode, or any other appropriate light emitter. In a non-limitative example, the light source is a high pressure mercury lamp used with a bandpass filter to select wavelengths between 340 nm and 420 nm. In another non-limitative example, the light source is a UV laser with a wavelength between 340 nm and 420 nm. Generally, the intensity of the light directed at the substrate 12 surface ranges from about 1 mW to about 1000 mW cm².

In an embodiment, programmable spatial optical modulators are used to generate light patterns for desired synthesis patterns. Non-limitative examples of a spatial optical modulator is a micromirror array modulator (DMD, which is commercially available from Texas Instruments, located in Dallas, Tex.). Other suitable means for projecting a light pattern are liquid crystal displays (LCD), liquid crystal light valves, acousto-optic scanning light modulators (SLMs), Galvanometric laser scanners, and/or the like.

The apparatus 100 and the methods disclosed herein advantageously allow the synthesis of more than one substrate simultaneously. Generally, this includes putting more than one substrate 12 into the reactor 102 and having a transparent substrate as the top cover of the reactor cartridge. Multiple arrays may be fabricated in parallel, either through a step and repeat exposure scheme or through a rotary turntable system.

It is to be understood that this multiplex synthesis system may have as few as two and as many as tens of substrates 12 processed simultaneously. In an example, 6-30 substrates 12 may be processed in a multiplex fashion mounted on a substantially linear X-Y translation stage.

In another embodiment, the substrates 12 on which synthesis is carried out are stationary, and the projected light pattern is moved from substrate 12 to substrate 12 in a programmed manner.

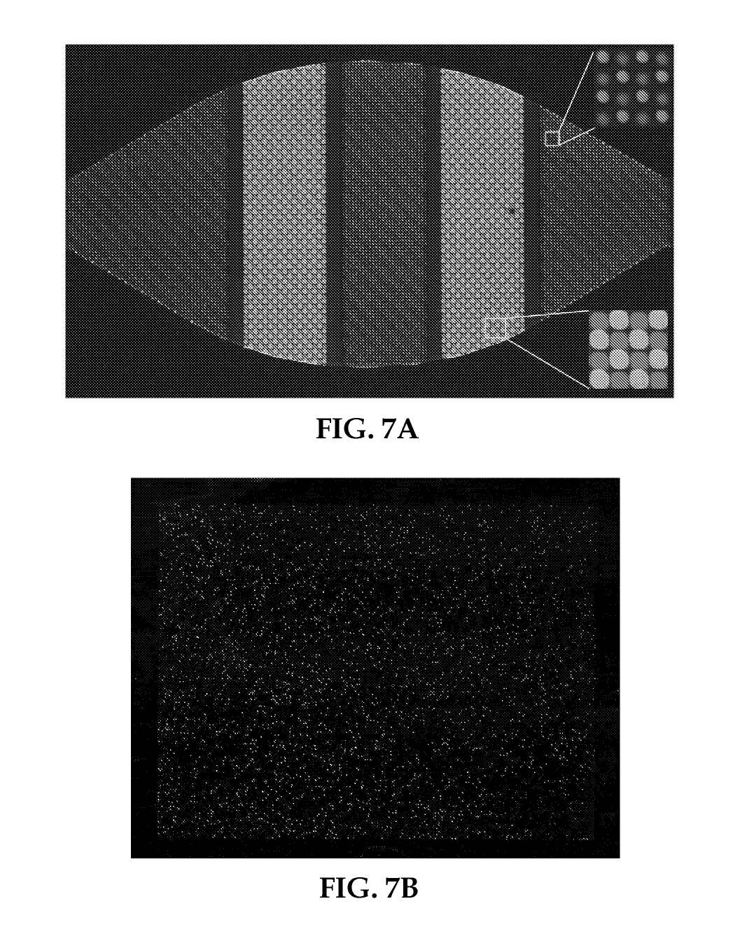

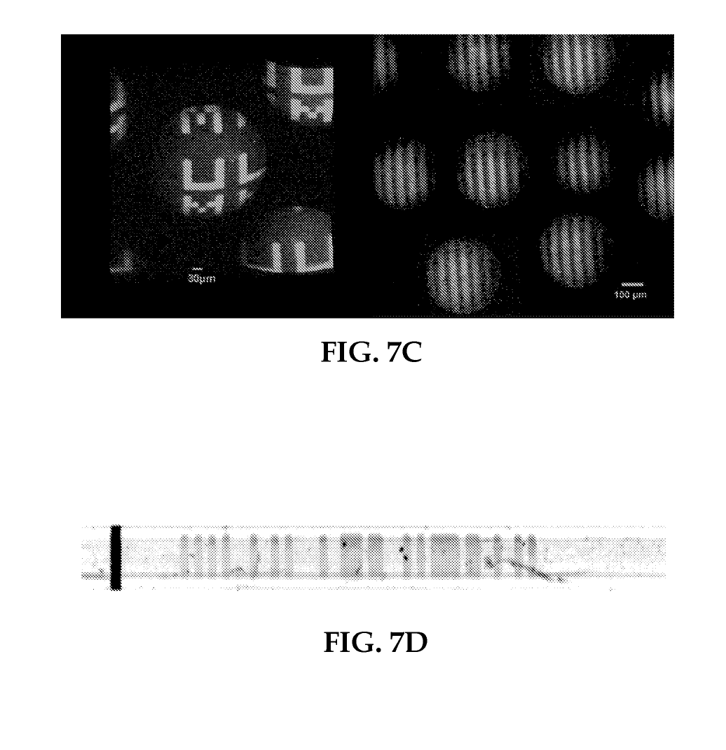

Referring now to FIGS. 7A-7D, the syntheses of oligonucleotide sequences 10 are shown on various substrates. FIGS. 7A and 7B depict the oligonucleotide sequences 10 on unpatterned glass slides. Oligonucleotides of various lengths (n=15-90) and various sequences (A, C, G, and T) are synthesized on a microscope slide using a photogenerated acid precursor and an embodiment of the method disclosed herein. The vertical bands shown in FIG. 7A are due to the projection used. FIG. 7C depicts oligonucleotides synthesized in the form of letters (left) and stripes (right) on a 200 micron sphere, and FIG. 7D depicts oligonucleotide synthesis in the form of a barcode on the inside surface of a 0.5 mm capillary. The dark bars are the DNA sequences.

While several embodiments have been described in detail, it will be apparent to those skilled in the art that the disclosed embodiments may be modified. Therefore, the foregoing description is to be considered exemplary rather than limiting.

What is claimed is:

1. A method for forming a molecular sequence, comprising:
derivatizing an unconfined substrate surface with at least one linker containing a protected reactive group;
contacting the substrate with a solution containing a photogenerated reagent precursor and at least one of a buffer or a neutralizer;
generating a photogenerated reagent in at least a portion of the solution, the photogenerated reagent configured to initiate the formation of at least one active region on the substrate surface; and
coupling a monomer to the at least one active region.

2. The method as defined in claim 1 wherein the photogenerated reagent precursor is selected from a photoacid generating molecule and a photobase generating molecule.

3. The method as defined in claim 2 wherein the photogenerated reagent is selected from an acid and a base.

4. The method as defined in claim 1 wherein generating the photogenerated reagent is accomplished via selectively exposing at least one region of the substrate to electromagnetic radiation.

5. The method as defined in claim 4 wherein the photogenerated reagent is substantially confined to the at least one exposed region via the at least one of the buffer or neutralizer.

6. The method as defined in claim 1 wherein the photogenerated reagent diffuses to a surface of the substrate where it catalyzes deprotection of the protected reactive group, thereby exposing a reactive group and forming the at least one active region.

7. The method as defined in claim 1 wherein prior to coupling the monomer to the at least one active region, the method further comprises removing the solution from the substrate.

8. The method as defined in claim 1 wherein the monomer has two ends, one of the two ends being unprotected and an other of the two ends having a protected reactive group attached thereto.

9. The method as defined in claim 8 wherein the contacting, the generating, and the coupling steps are repeated to form a predetermined sequence.

10. The method as defined in claim 1 wherein the solution further comprises at least one of a sensitizer, a stabilizer, a viscosity additive, or combinations thereof.

11. The method as defined in claim 1 wherein the photogenerated reagent is an acid, and wherein the buffer or neutralizer is selected from pyridine, lutidine, piperidine, primary amines, secondary amines, tertiary amines, derivatives thereof, and combinations thereof.

12. The method as defined in claim 1 wherein the photogenerated reagent is a base, and wherein the buffer or neutralizer is selected from benzillic acid, aluminum chloride, iron (III) chloride, boron trifluoride, ytterbium (III) triflate, butyric acid, propionic acid, phenol, and combinations thereof.

13. The method as defined in claim 1 wherein the monomer has two unprotected ends.

14. The method as defined in claim 1 wherein the monomer is in a solution containing at least one activator.

15. The method as defined in claim 14 wherein the at least one activator is selected from tetrazole; 4,5,dicyanoimidazole (DCI); pyridiniumtrifluoroacetate; 5-ethlythiotetrazole; (3,5-dintrophenyl)-1H-tetrazole; trimethylchlorosilane; activator 42 (5-(bis-3,5-trifluormethylphenyl)1-H-tetrazole; derivatives thereof, and combinations thereof.

16. A molecular sequence formed by the method of claim 1.

17. An apparatus, comprising:
an unconfined substrate surface; and
a molecular sequence coupled to the unconfined substrate surface.

18. The apparatus as defined in claim 17 wherein the molecular sequence includes a first monomer coupled to the unconfined substrate surface via a reactive group of a linker.

19. The apparatus as defined in claim 18 wherein the reactive group initially contains a protection group that is removed via a photogenerated reagent.

20. The apparatus as defined in claim 17 wherein the molecular sequence is formed of a plurality of monomers coupled together.

21. The apparatus as defined in claim 20 wherein each of the plurality of monomers is selected from nucleotides, locked nucleic acid monomers, amino acids, monosaccharides, disaccharides, and combinations thereof.


SEQUENCE LISTING (12 sequences)

SEQ ID NO: 1 - DNA, 9 bp, Artificial Sequence
  OTHER INFORMATION Synthetic
accgtatga

SEQ ID NO: 2 - DNA, 9 bp, Artificial Sequence
  Synthetic
taggtagtc

SEQ ID NO: 3 - DNA, 9 bp, Artificial Sequence
  Synthetic
tatcgctaa

SEQ ID NO: 4 - DNA, 9 bp, Artificial Sequence
  Synthetic
actcgagct

SEQ ID NO: 5 - DNA, 9 bp, Artificial Sequence
  Synthetic
atcgcatta

SEQ ID NO: 6 - DNA, 4 bp, Artificial Sequence
  Synthetic
cgat

SEQ ID NO: 7 - DNA, 4 bp, Artificial Sequence
  Synthetic
tcac

SEQ ID NO: 8 - DNA, 4 bp, Artificial Sequence
  Synthetic
gact

SEQ ID NO: 9 - protein, 4 aa, Artificial Sequence
  Synthetic
HKAV

SEQ ID NO: 10 - protein, 4 aa, Artificial Sequence
  Synthetic
SHAR

SEQ ID NO: 11 - protein, 4 aa, Artificial Sequence
  Synthetic
KARV

SEQ ID NO: 12 - protein, 5 aa, Artificial Sequence
  Synthetic
AHVSK